SEQUENCE LISTING (16 sequences)

SEQ ID NO: 1 - DNA, 22 bp, Mus musculus
tcgagctgctctatcccatgat

SEQ ID NO: 2 - DNA, 20 bp, Mus musculus
gggtctatcgctggcagtca

SEQ ID NO: 3 - DNA, 23 bp, Mus musculus
gacctcttctcaacagccacttc

SEQ ID NO: 4 - DNA, 23 bp, Mus musculus
tccttctccaaacgcatctctga

SEQ ID NO: 5 - DNA, 25 bp, Mus musculus
tcttggcactgtgtactacataact

SEQ ID NO: 6 - DNA, 25 bp, Mus musculus
gcaggaaggatacaccatctttaac

SEQ ID NO: 7 - DNA, 23 bp, Mus musculus
ggttggaagaaagggtgtcatca

SEQ ID NO: 8 - DNA, 23 bp, Mus musculus
ggacaatggactgaacactggaa

SEQ ID NO: 9 - DNA, 23 bp, Mus musculus
ggactgacacaggtgaaccaatg

SEQ ID NO: 10 - DNA, 24 bp, Mus musculus
tccttatctcgttccttctccaca

SEQ ID NO: 11 - DNA, 22 bp, Mus musculus
attgaccaggaggagctgaaca

SEQ ID NO: 12 - DNA, 24 bp, Mus musculus
accttctacagagaagtgcttgac

SEQ ID NO: 13 - DNA, 23 bp, Mus musculus
gtggagtagcaagacagagactg

SEQ ID NO: 14 - DNA, 24 bp, Mus musculus
ttcataagttcccaatcccacaca

SEQ ID NO: 15 - DNA, 20 bp, Mus musculus
ggagatcaaccccaggcaca

SEQ ID NO: 16 - DNA, 24 bp, Mus musculus
gctgctatcatggcattctcatag

(19) United States
(12) Patent Application Publication
(10) Pub. No.: US 2015/0196606 A1
(43) Pub. Date: Jul. 16, 2015

(54) ACTIVATOR OF GENE EXPRESSION OF MOLECULAR CHAPERONE GENE COMPRISING EGGSHELL MEMBRANE COMPONENT AND COMPOSITION THEREOF

(71) Applicants: ALMADO Inc., Tokyo (JP); The University of Tokyo, Tokyo (JP)

(72) Inventors: Yukio HASEBE, Tokyo (JP); Junko ATOMI, Tokyo (JP); Miho SHIMIZU, Tokyo (JP)

(21) Appl. No.: 14/673,244

(22) Filed: Mar. 30, 2015

Related U.S. Application Data
(63) Continuation of application No. 14/092,175, filed on Nov. 27, 2013.

(30) Foreign Application Priority Data
Jun. 11, 2013 (JP) 2013-122528

(51) Int. Cl. A61K 35/57; A61K 9/16; A61K 45/06

(52) U.S. Cl. CPC A61K 35/57; A61K 45/06; A61K 9/16

(57) ABSTRACT

To provide an activator of gene expression of molecular chaperone gene and applications thereof.

This activator of gene expression of molecular chaperone gene contains an eggshell membrane component, for example, a powder containing eggshell membrane or a soluble eggshell membrane component. Moreover, the powder containing eggshell membrane used in the present invention may be a fine powder preferably having 6 μm or less in the mean volume particle diameter of the fine powder containing eggshell membranes, and/or 20 μm or less in the maximum volume particle diameter thereof, but not limited to these values.

### CROSS REFERENCE TO RELATED APPLICATIONS

The present application is a continuation application of U.S. patent application Ser. No. 14/092,175, filed on Nov. 27, 2013, the entire contents of which are incorporated herein by reference and priority to which is hereby claimed. Priority under 35 U.S.C. §119(a) and 35 U.S.C. §365(b) is hereby claimed from Japanese Application No. 2013-122528 filed Jun. 11, 2013, the entire content of which is incorporated herein by reference.

### BACKGROUND

This application is based upon and claims the benefit of priority from Japanese Patent Application No. 2013-122528, filed on Jun. 11, 2013, the disclosure of which is incorporated herein in its entirety by reference.

1. Technical Field

The present invention relates to an activator of gene expression of molecular chaperone comprising an eggshell membrane component, for example, a soluble eggshell membrane component (eggshell membrane hydrolysates and the like) or powders or fine powders containing eggshell membrane, and applications thereof.

2. Background Art

An eggshell membrane (hereinafter sometimes referred to as “ESM”) is a membrane that is found inside the eggshell of eggs of avian species such as chicken and the like. It has an antibacterial activity and anti microorganism activity, and protects an embryo from infection during the embryonic development. The eggshell membrane has a netlike structure comprising a strong fiber-type protein, which contains I-type, V-type, and X-type collagens, glucosamine, desmosine and hyaluronic acid as main components. Many of these proteins contain cysteine, and are relatively stable to acids, alkalis, and proteases, and insoluble to water. Therefore, eggshell membranes of chicken eggs have not been utilized and discarded as by-products in the food industry. However, it has been known that they have actions to promote skin regeneration, in particular, generation of III-type collagen, which is referred to as fatal collagen. Therefore, the effectiveness for living organisms has been paid attention.

It has been known that collagens in the skin decrease with age. The inventors reported that the expression levels of III-type collagen, decorin, and matrix metalloproteinase 2 (hereinafter referred to as “MMP2”) in human skin fibroblast cells cultured on alkali-hydrolyzed eggshell membranes (hereinafter sometimes referred to as “ASESM”) bound to an artificial polymer, which were close to the dermis (under the condition, cells are sparsely present), were significantly higher compared to those in human skin fibroblast cells cultured on a collagen coat or a cell culture dish in the same condition (NPL 1: Ohto-Fujita et al, Cell Tissue Res. 2011 July; 345(1): 177-190)

It has been reported that aging in “physical appearance” such as wrinkles of the facial skin that become obvious with age, and the depression of the brain/skeletal muscle functions that appear in cognition/movement and the like are associated to the onset of the aggregation of denatured proteins and the production of toxicity in a cell due to the declining of homeostatic control mechanism that intrinsically exists in a body. For the normalization of these conditions, molecular chaperones that have the activity of helping the folding of other proteins, for example, various heat shock proteins (HSPS: HSP90, HSP70, HSP60, HSP40 (numbers indicate molecular weight (kDa)) and low-molecular weight HSP (sHSP)) are associated. There are HSP25/27 (HSPB1), MKBP (HSPB2), Alpha-B crystallin (cryab, HSPB5, heat shock protein beta 5), Hsp20 (HSPB6, heat shock protein beta 6), HSP22 (HSPB8, heat shock protein beta 8) as sHSP in the brain. And it has become clear that the overexpression of these proteins play a protective role in neurological degeneration (NPL 2: Brownellet al., 2012; Front Immunol. 2012; 3:74.doi: 10.3389/fimmu. 2012. 00074). The inventors previously reported that HSPB5 acted as a protective molecule for atrophy of slow muscles (NPL 3: Atomi et al, 1991, Atomi, Y., Yamada, S., & Nishida, T., Early changes of alpha B-crystallin mRNA in rat skeletal muscle to mechanical tension and denervation. Biochem Biophys Res Commun 181, 1323-1330.; Atomi, Y., Yamada, S., Strohman, R., & Nonomura, Y., Alpha B-crystallin in skeletal muscle: purification and localization. J Biochem (Tokyo) 110, 812-822. (1991); NPL 4: Sakurai T, Fujita Y, Ohto E, Oguro A, Atomi Y. The decrease of the cytoskeleton tubulin follows the decrease of the associating molecular chaperone alpha B-crystallin in unloaded soleus muscle atrophy without stretch. FASEB J. 2005 July; 19(9): 1199-201. Epub 2005 May 13. PubMed PMID: 15894563). It has been found that αB is expressed in crystalline lens, heart, brain and skin.

However, the activities of mechanism of eggshell membrane components or the efficient methods to enhance the activities of molecular chaperones were not clearly understood. Also, substances that contribute to physical and emotional health by controlling a plurality of molecular chaperone genes in the skin at the same time have not yet been known.

### SUMMARY OF INVENTION

### Technical Problem

The present invention is invented in light of above-mentioned circumstances, and a problem in the present invention is to provide an activator of gene expression of molecular chaperone gene, which can be employed with a greater safety profile in a simplified method of acting on cells on a routine basis. Especially, it is to provide a plurality of activators of gene expression of molecular chaperone genes at the same time, the applications of medicine and/or cosmetic product compositions, food products (supplements), food additives, which have multiple effects by a single material maintaining and/or improving body in good condition.

### Solution to Problem

The inventors have found that an eggshell membrane component acts on skin cells and the like, and activates various genes encoding molecular chaperone, and have achieved to complete the present invention.

Accordingly, the above-mentioned problems are achieved by the present invention. Namely, the activator of gene expression of molecular chaperone gene of the present invention contains an eggshell membrane component, in particular, powders containing eggshell membranes or a soluble eggshell membrane component (for example, hydrolysates of eggshell membranes).

In one embodiment of the activator of gene expression of molecular chaperone gene used in the present invention, powders containing eggshell membranes are fine powders and preferably the mean volume particle diameter of the fine powders containing eggshell membranes is 6 μm or less and/or the maximum volume particle diameter is 20 μm or less.

One embodiment of the activator of gene expression of molecular chaperone gene used in the present invention is a regulator of the expression of one or more genes of Hspb5 (Cryab, crystallin, alpha B), Hspa1b (Hsp70-1, heat shock protein 70), Hspd1 (Hsp60, heat shock 60 kDa protein 1 (chaperonin)), Hsp90aa1 (heat shock protein 90, alpha (cytosolic), class A member 1), Hsp90ab1 (heat shock protein 90 kDa alpha (cytosolic), class B member 1), Hsp90b1 (heat shock protein 90, beta (Grp94), member 1), Trap1 (TNF receptor-associated protein 1) and Hspb2.

One embodiment of the activator of gene expression of molecular chaperone gene used in the present invention is preferably used as at least one or more intended purposes of compositions for oral use, compositions for external use, food products (supplements), food additives, for regenerative medicine (stem cell, iPS cell, and the like), and materials for bases.

A composition that is used for body in the present invention such as medicine or cosmetic product compositions and the like preferably contains a diluting agent with the activator of gene expression of molecular chaperone gene of the present invention. In that case, it is preferable to prepare compositions for external use or compositions for oral to improve general condition of the entire body. As the eggshell membrane component, a soluble eggshell membrane component is preferably used for the compositions for external use, and powders containing eggshell membranes are preferred for the compositions for oral use. In one embodiment of a pharmaceutical composition in the present invention, tablets are preferable.

In other embodiment of the compositions for oral use in the present invention, the eggshell membrane component is preferably contained at a ratio ranging from 5 to 40%. In the embodiment of the composition for external use, the soluble eggshell membrane component can be contained at a ratio ranging from 1 to 80%, preferably at a ratio ranging from 1 to 40%.

A food additive in the present invention consists the activator of gene expression of molecular chaperone gene in the present invention, or contains it. Moreover, food products of the present invention are characterized by adding this food additive.

### Advantageous Effects of Invention

According to the present invention, an activator of gene expression of molecular chaperone gene comprising an eggshell membrane component containing soluble eggshell membranes or powders containing eggshell membranes, and an application thereof can be provided. According to the activator of gene expression of molecular chaperone gene in the present invention, a mean with none or extremely low risk of side effects and extreme safety for controlling and moderately activating the expression of molecular chaperone genes can be provided. The eggshell membranes of chicken, which are generally discarded, can be effectively utilized to produce the activator of gene expression of molecular chaperone gene in the present invention without the requirement of cumbersome procedures. Therefore, it can be easily produced with a high yield, and advantageous from the point of view of being economic and the protection of environment.

Moreover, the activator of gene expression of molecular chaperone gene in the present invention can be widely applicable for medical supplies such as functional foods, medicine for prophylactic agents/therapeutic agents and the like by converting it into an appropriate composition depending on applicable objects and purposes. The compositions in these aspects can be easily utilized routinely by means such as applying or ingesting and the like, and be easily improved in terms of mechanical, physical, and chemical properties of the skin such as the water content/elasticity and the like in safety. The activator of gene expression of molecular chaperone gene in the present invention can activate or regulate a plurality of genes encoding molecular chaperone systemically including the skin at the same time. Therefore, the activator of gene expression of molecular chaperone gene can provide (1) maintenance/improvement of good health through the activation of the entire body by the moderate promotion of the expression of molecular chaperone genes; (2) improvement of the slight deviation from normal range of homeostasis such as “distemperature in some vague way”; (3) direct improvement of diseases of target cells/tissues; (4) early recovery from diseases and prevention of side effects resulted from drugs such as antitumor drugs by a concomitant use of various treatments utilizing its conditioning effect in medical practice; (5) a buffer function of improving several physical illnesses at the same time; and (6) effects such as improving the prevention and recovery from injuries due to physical fatigues such as sports. Furthermore, the activator of gene expression of molecular chaperone gene in the present invention has no irritability and can be concomitantly used with various components in various embodiments. Therefore, it can be concomitantly used with other nourishments (food products), beauty components to obtain further effects for the entire body, and can be applied for a specified composition with a specific effect.

### BRIEF DESCRIPTION OF DRAWINGS

FIG. 1 shows the expression of the molecular chaperone gene on the dorsal region of a mouse. The left panel A shows the expression of Hspb5. The right panel B shows the expression of Hspb2 (HSP27). In each group, “cont.” represents a control group; “ESM+” represents a group administered with fine powders containing eggshell membranes. Fine powders containing eggshell membranes were orally administered to mice at a single-dose, and then the levels of the gene expression were measured as the amount of mRNA (a relative quantity when defined the expression of GAPDH as 1) (n=3).

FIG. 2 shows the radioactivity concentration over the time course of a part of tritium-labeled powders containing eggshell membranes in the blood that is digested and absorbed after oral administration to a mouse.

FIG. 3 shows the radioactivity concentrations of the tritium-labeled powders containing eggshell membranes, which is digested and absorbed, in each tissue 2, 6 and 12 hours after oral administration to a mouse.

### DESCRIPTION OF EMBODIMENTS

### (Activator of Gene Expression of Molecular Chaperone Gene)

The activator of gene expression of molecular chaperone gene in the present invention contains an eggshell membrane component as an active ingredient. The eggshell membrane component may include the eggshell membrane itself, any processed products or extraction of the eggshell membranes and the like. For example, powders containing eggshell membranes or a soluble eggshell membrane component (hydrolysates and the like) can be utilized.

For the eggshell membrane that is consisted of the eggshell membrane component used in the present invention, the inside membranes of the eggshell of any eggs of terrestrial oviparous animals, in particular, of eggs of avian species (outer eggshell membranes and/or inner eggshell membranes and/or limiting membranes) can be used. Among them, in particular, the eggshell membranes of a chicken egg are preferably used in terms availability, cost, and the like.

### (The Soluble Eggshell Membrane Component Used in the Present Invention)

The eggshell membrane component used in the present invention can be a soluble component of the eggshell membrane, for example, degradation products or extraction of the eggshell membranes. A hydrolysate of the eggshell membranes can be produced by publicly known methods and the modified methods thereof, for example, a method of a soluble eggshell membrane characterized by degrading eggshell membranes in an alkali aqueous organic solvent, neutralizing the obtained degraded solution, and filtrating (Japanese Patent Application Publication (JP-B) No. H06-21047); a method of producing water-soluble eggshell membranes characterized by treating with a proteolytic enzyme (JP-B No. H07-110210); a treatment method with an anion-exchange resin in an alkali aqueous organic solvent after hydrolysis (Japanese Patent No. 5179847); and an alkali hydrolysis method disclosed such as in U.S. Pat. No. 8,211,477 (title of invention: Solubilized protein composition obtained from eggshell membrane).

Methods other than alkali hydrolysis methods, a soluble eggshell membrane can be produced, for example, based on;

a picric acid-pepsin treatment (Takahashi K, Shirai K, Kitamura M, Hattori M. Soluble eggshell membrane protein as a regulating material for collagen matrix reconstruction. Biosci Biotechnol Biochem. 1996 August; 60(8): 1299-302)),

an acid-pepsin hydrolysis method (F. Yi, J. Yu, Z. X. Guo, L. X. Zhang, and Q. Li, “Natural bioactive material: a preparation of soluble eggshell membrane protein,” Macromolecular Bioscience, vol. 3, no. 5, pp. 234-237, 2003; F. Yi, Z. X. Guo, L. X. Zhang, J. Yu, and Q. Li, “Soluble eggshell membrane protein: preparation, characterization and biocompatibility,” Biomaterials, vol. 25, no. 19, pp. 4591-4599, 2004; Jun Jia, Geng Liu, Jian Yu, and Yuanyuan Duan. 2012. Preparation and characterization of soluble eggshell membrane protein/PLGA electrospun nanofibers for guided tissue regeneration membrane. J. Nanomaterials 2012, Article 25 (January 2012), 1 pages. DOI=10.1155/2012/282736 http://dx.doi.org/10.1155/2012/282736)),

a method of reduction of S—S bond and trypsin treatment (Kodali V K, Gannon S A, Paramasivam S, Raje S, Polenova T, Thorpe C. A novel disulfide-rich protein motif from avian eggshell membranes. PLoS One. 2011 Mar. 30; 6(3): e18187. doi: 10.1371/journal.pone.0018187), and the like.

The soluble eggshell membrane component may be produced by these methods using powders containing eggshell membranes as described below instead of the eggshell membranes.

Commercially available materials can be employed as the degradation products of the eggshell membranes. For example, an eggshell membrane hydrolysate produced by Kewpie (Kewpie Corporation, Tokyo, Japan), of which trade name is “EM PROTEIN-P”, can be used.

(Powders containing Eggshell Membranes Used in the Present Invention)

Powders containing eggshell membranes used in the present invention are preferably fine powders containing eggshell membranes having 6 μm or less in the mean volume particle diameter, but it is not particularly limited as long as the powders contain at least the eggshell membranes. Moreover, the fine powders containing eggshell membranes used in the present invention preferably has 20 μm or less in the maximum volume particle diameter. However, the terms “mean volume particle diameter” and “maximum volume particle diameter” in the present description mean values that are measured by using a laser diffraction particle size distribution analyzer (LMS-30, Seishin Enterprise Co., Ltd.). Here, term “mean volume particle diameter” means a particle size when a cumulative value from the side of small particle size in particle size distribution is 50%. Moreover, in the case of measurement of the particle size of powders or fine powders containing eggshell membranes, a sample for measurement dispersed powders or fine powders containing eggshell membranes in water using a surfactant is used. Term “powder” means any powdery materials despite of particle sizes, term “fine powder” means a powder of which a maximum particle size and/or average particle size is less than about 100 μm, but it is not intended restrict differentiation.

Furthermore, the efficiency of digestion and absorption and activation efficiency of the gene encoding molecular chaperone can be further improved by controlling the particle size distribution of the fine powders containing eggshell membranes having 6 μm or less in the mean volume particle diameter or 20 μm or less in the maximum volume particle diameter, compared to the conventional eggshell membrane powders obtained by the classifying treatment with a 70 mesh or 150 mesh (eggshell membrane powders at a maximum particle size ranging from 100 to 200 μm).

The reasons associated with these effects are not well known, however, it can be extrapolated as follows. In general, as a particle size becomes smaller, the surface area in a unit volume of the particle increases. Therefore, if particle comprises only a material that is soluble or easily soluble to digestive juice, as particle size undergoes smaller, the efficiency of digestion and absorption is improved. As a result, the activation efficiency of gene expression of molecular chaperone gene is expected to increase.

However, regarding conventional eggshell membrane powders with ranging from 100 to 200 μm in the maximum particle size, several tens to a hundred and several tens μm in the average particle size, even though the maximum particle size or average particle size alter within the range of these particle sizes, and the particles are further ground to get smaller particles, the efficiency of digestion and absorption and the efficiency of gene activation of molecular chaperone gene are rarely improved. The reason for this is thought that the eggshell membrane has a robust netlike structure consisting of a fibrous protein as a main component, and the robust netlike structure is still retained in the eggshell membrane particles that are ground in the range of the particle size mentioned above.

By contrast, when fine powders containing eggshell membranes having 6 μm or less in the mean volume particle diameter or 20 μm or less in the maximum volume particle diameter are used, the efficiency of digestion and absorption and the gene activation of molecular chaperone gene are substantially improved respectively compared to the conventional eggshell membrane powders. It is speculated that such improvement of the efficiency of digestion and absorption and the gene activation of molecular chaperone gene would not be simply attributable to small particle size. But it would be attributable that the robust netlike-structure containing fibrous property, which eggshell membranes intrinsically possess, is destroyed in the whole fine particles of eggshell membrane in the process of pulverization of eggshell membranes and the whole fine particles of eggshell membrane become easily soluble to digestive juice.

Therefore, in order to use the powders as the eggshell membrane component in the present invention, the maximum volume particle diameter may exceed 20 μm; the mean volume particle diameter may exceed 6 μm; and the maximum volume particle diameter may exceed 20 μm and the mean volume particle diameter may exceed 6 μm. However, in light of further improvement of the efficiency of the digestion and absorption and activation efficiency of gene expression of the molecular chaperone gene, the fine powders containing eggshell membranes have preferably 6 μm or less in the mean volume particle diameter and/or 20 μm or less in the maximum volume particle diameter.

The activator of gene expression of molecular chaperone gene comprising the powders containing eggshell membranes in the present embodiment contains at least an eggshell membrane component that is powderized or pulverized, and may contain an eggshell calcium component that is powderized or pulverized. In this instance, the powder containing eggshell membranes in the present embodiment is particularly preferably either an embodiment containing only the eggshell membrane component (the first embodiment) or an embodiment containing the eggshell membrane component and the eggshell calcium (the second embodiment). In the case of the activator of gene expression of molecular chaperone gene comprising powders containing eggshell membranes in the first embodiment, the agent purely contains the eggshell membrane component alone. Therefore, they can be widely utilized in various applications, namely pharmaceutical compositions, in particular, pharmaceutical compositions such as solid dosage forms including tablets and the like, food additives, and the like. In either the powders containing eggshell membranes in the first embodiment or the powders containing eggshell membranes in the second embodiment, the contamination of impure substances in the process of production may be permitted. Moreover, the activator of gene expression of molecular chaperone gene comprising the powders containing eggshell membranes in the present embodiment may contain other nutritive components other than the eggshell membrane component and the eggshell calcium component.

(A Method of Producing Powders or Fine Powders containing Eggshell Membranes Used for the Activator of Gene Expression of Molecular Chaperone Gene in the Present Invention)

To produce the powders containing eggshell membranes used in the present invention, a raw material that are stripped eggshell membranes or a raw material that is in the condition of eggshell membranes attaching to eggshell may be used, and the raw material and eggshell membrane powders can be concomitantly used. Such raw material may be powderized in any publicly known methods. Commercially available eggshell membrane powders may be used as powders containing eggshell membranes. Examples of the commercially available eggshell membrane powders include, for example, a trade name of “EM powder 300” (Kewpie Corporation) can be used. When fine powders containing eggshell membranes are produced, commercially available eggshell membrane powders, or commercially available eggshell membrane powders and eggshell calcium may be used and further pulverized up to 6 μm or less in the mean volume particle diameter, and/or, 20 μm or less in the maximum volume particle diameter.

The fine powders containing the eggshell membranes used in the present invention can be produced at least through a process of pulverization, in which the raw materials containing the eggshell membranes are clashed in a gas to pulverize. In such a pulverization process, so-called a jet mill is used. Such a grinding method does generate less friction heat caused by contact and collision between a crashing member and raw materials during pulverization compared to conventional methods in which hard crashing members such as a rotary blade collides with raw materials and grind. Therefore, it gives less damage to components such as amino acids and proteins contained in the eggshell membranes, which are easily denatured, deteriorated, and decomposed. That is, an active ingredient in the eggshell membranes becomes hardly lost in the production process. In addition to this, high-pressure gas instead of the crashing member is used in order to grind raw materials, so that impure substances derived from grinding equipment do not contaminate with fine powders containing eggshell membranes, which is advantageous.

In the process of pulverization, the raw materials containing eggshell membranes are preferably ground with a jet mill until the mean volume particle diameter becomes 40 μm or less, more preferably 20 μm or less, further preferably 10 μm or less. Moreover, in this instance, the raw materials are preferably ground until the maximum volume particle diameter becomes 20 μm or less. The lower limit of the mean volume particle diameter of the raw materials containing eggshell membranes ground with a jet mill is not particularly limited, but 4 μm or more is preferable, and 5 μm or more is more preferable in terms of practical uses such as productivity and the like.

The raw materials containing eggshell membranes after grinding with the jet mill can be used as it is for the activator of gene expression of molecular chaperone gene comprising fine powders containing eggshell membranes in the present invention, when the maximum volume particle diameter is 20 μm or less, and/or, the mean volume particle diameter is 6 μm or less. When coarse particles exceeding 20 μm in particle size are contained in the particle size distribution, a process of classification to remove coarse particles by sorting with a sieve having 20 μm or less sieve opening may be further conducted after the pulverization process.

Moreover, in the method of producing fine powders containing eggshell membranes used for the activator of gene expression of molecular chaperone gene in the present invention, other steps/processes may be carried out when needed. For example, the process of pulverization may contain the first pulverization treatment and the second pulverization treatment, and the second pulverization treatment may be conducted after the raw material powders treated with the first pulverization is sterilized with high-pressure steam. In the process of pulverization of raw materials containing eggshell membranes with the jet mill, antibacterial activity of the eggshell membrane tends to decrease. However, the propagation of mold and bacteria on fine powders containing eggshell membranes in the present embodiment can easily be prevented by the sterilization treatment as mentioned above.

(A Composition containing the Activator of Gene Expression of Molecular Chaperone Gene)

The composition of the present invention contains at least one type of a diluting agent along with the activator of gene expression of molecular chaperone gene in the present invention. Since the activator of gene expression of molecular chaperone gene in the present invention is free from irritable properties, the formulations thereof are not particularly limited when medicine or cosmetic products, and the like are prepared from the composition. Thus, any compositions for oral use or external use can be prepared. Compositions for external use such as ophthalmic preparations, nose drops, ear drops, agents for oral use (oral rinse, spray), suppositories (suppository, ointment, enema clyster), and the like are prepared in various dosage forms such as liquid formulations, solid preparations, semi-solid preparations, and the like according to intended purposes by mixing commonly-used publicly known components. Preferable compositions, for example, include lotion, ointment, gel, cream, spray, adhesive skin patches, powders, and the like. For the intended purpose of oral administration or uptake, compositions for oral use such as tablets, powders, granules, encapsulated formulations, and liquid formulations are preferable. Compositions for oral use maybe sublingual tablets (not only tablets but also sheets such as oblate and paste), jelly, and drinkable preparations suspended with fine powders. In terms of absorption from mucous membrane in the oral cavity, the active ingredient can directly reach to the heart from capillary vessels via the internal jugular vein, thus, the degradation and metabolism in gastrointestinal tract and the first-pass effect caused by metabolism in the liver can be avoided, so that it is advantageous to circulate it throughout the entire body at once. Various components and methods to produce compositions such as medicine or cosmetic products in various formulations containing components mentioned above are publicly known in the art of production of medicine, cosmetic products and the like. And persons having ordinary skills in the art can arbitrarily select the production method when needed. Herein, a “pharmaceutical composition” is not limited to the composition for human, and includes pharmaceutical compositions for mammals such as dogs and cats that are reared as pets and livestock. Moreover, a “cosmetic product composition” includes not only cosmetic products but also various quasi-drugs and medicinal cosmetic products specified by the Pharmaceutical Affairs Act in Japan.

In the present description and Claims, “%” means percentages when the total weight or volume of the total composition is defined as 100%, unless otherwise particularly specified. Further, basically (W/V) or (W/W) is used when the objective component is in a solid state (powders and the like), and (V/V) is used when the objective component is in a liquid form.

The effective dosage amount of the pharmaceutical composition for gene activation of molecular chaperone in the present invention varies depending on the types and degrees of diseases that should be treated or prevented, the condition of subjects for administration (including age, gender, physical conditions, and the like), and formulations.

The oral dosage amount of such a pharmaceutical composition for human (adult with a body weight of 60 kg), is preferably in a range from 1 mg to 100,000 mg/day converting to the amount of the eggshell membrane component. Specifically, the dosage amount of the pharmaceutical composition for oral use in the present invention can be adjusted to in a range from 18 to 48,000 mg of eggshell membrane component in total per day, and further preferable value is in a range from 35 mg to 3,500 mg.

Moreover, in the case of composition for external use, although it is different depending on the area of the skin and regions for application, pharmaceutical compositions for external use containing eggshell membrane component can be applied at a dose of about 1 to 400 mg/ml (0.1 to 40%) as the amount of eggshell membrane component dividing into one to several times per day. Method of applying is not limited to application, and can be arbitrarily selected. For example, the composition may be a spray as liquid form and adhesive skin patch as film-like form.

The activator of gene expression of molecular chaperone gene in the present invention is extremely safe and has no concerns about side effects, so there is not issue even though amount of administration or application exceeds the range mentioned above as long as other components are appropriately chosen to produce compositions.

### (Compositions for External Use)

In order to prepare agents for topical applications, according to their objectives for use, by the incorporation of the activator of gene expression of molecular chaperone gene in the present invention to publicly known components that is commonly-used, various dosage forms thereof such as liquid formulation, solid preparation, semi-solid preparation and the like can be prepared. For the composition for external use in the present invention, in addition to the activator of gene expression of molecular chaperone gene and diluting agent in the present invention, for example, active ingredients for the purpose of cosmetics or medicine, fragrance ingredients (flavor and the like), colorants and the like can be employed. Examples of other active ingredients include, for example, antiphlogistic agents, anti-inflammatory drugs, melanin production suppressants, melanin reducing agents, hair bleaching agents, melanin excretion promoters, cell activators, antioxidants, anti-oxidizing agents, keratolytic-release agents, sebum suppressors, moisturizing agents, emollient agents, sebum secretion suppressors/promoters, ultraviolet absorbing agents, antiperspirants, blood circulation improving agents, exfoliating cleansers/softening agents, skin whitening agents, anti-allergic drugs, steroid hormones, immunosuppressant, antibiotics, and the like.

For example, a pharmaceutical composition or cosmetic product for improving water content in the skin and/or elasticity or improving general condition can be prepared by mixing the activator of gene expression of molecular chaperone gene in the present invention with one or more components such as carbon hydride (vaseline and the like), higher fatty acid lower alkyl ester (stearyl alcohol, isopropyl myristate, and the like), animal oil and fat (lanolin and the like), polyhydric alcohol (glycerin and the like), surfactant (glycerin fatty acid ester, monostearate polyethylene glycol, and the like), mineral salt, wax, resin, water, preservatives (methyl parahydroxybenzoate, butyl parahydroxybenzoate, and the like), peptides (acetyl hexapeptide-3, palmitoyl pentapeptide-4 (Matrixyl), and the like), sodium acetylated hyaluronate, caprylyl glycol, and the like.

The composition for external use in the present invention preferably contains a moisturizing ingredient and/or component for increasing viscosity is increased when an aqueous composition thereof is prepared. Examples of base moisturizing ingredients include, for example, glycerin, diglycerin, polyglycerin, propylene glycol, dipropylene glycol, 1-3 butylene glycol, hexylene glycol, maltitol, mannitol, sorbitol, xylitol, trehalose, sodium pyrrolidone carboxylic acid, sodium polyglutamate, sodium lactate, sodium polylactate, polyethylene glycol, saccharides, methyl glucoside, and the like. Examples of component for increasing viscosity include, for example, hyaluronic acid sodium, dermatan sulfuric acid sodium, dextrin, sodium alginate, carrageenan, xanthane gum, cornstarch, Gum Tragacanth, casein, polyvinyl alcohol, polyvinyl pyrrolidone, methyl cellulose, hydroxypropyl cellulose, xylan, mannan, galactan, pectin, extensin, gum arabic, pullulan, sodium polyacrylate, carboxy vinyl polymer, clay minerals, and the like. Moreover, 2-methacryloyloxyethyl phosphorylcholine (MPC) polymer is preferable since it gives environment that is close to the skin in fibroblast. 1-3 Butylene glycol, a moisturizing ingredient of the base, is preferably contained in compositions for external use; however, there are cases that are not preferable due to the association with allergy. Therefore, the ingredients are arbitrarily selected depending on symptoms.

### (Compositions for Oral Use)

The activator of gene expression of molecular chaperone gene in the present invention can be prepared in the form of compositions for oral use such as tablets, powders, granules, encapsulated formulation, liquid formulation, and the like. Various components and production methods to produce various formulations of compositions for oral use are publicly known methods of producing in the art of production of medicine and cosmetic products. And persons having ordinary skills in the art can arbitrarily chose the components and production methods when needed.

The composition for oral use in the present embodiment preferably contains at least one type of material selected from (1) health-enhancing agents (for example vitamins, β-carotene, royal jelly, and the like), (2) various components for medicinal use that can be combined (for example, anti-inflammatory drug and the like), in addition to a diluting agent.

Types of vitamins that are contained in the composition for oral use in the present embodiment are not particularly limited, and may be any as long as vitamins can be taken by human or mammals. For example, lipophilic vitamins such as vitamin A, vitamin D, vitamin E, vitamin F, vitamin K; and water-soluble vitamins such as vitamin B, vitamin C, vitamin H, vitamin L can be recited. The tablet in the present embodiment can contain one or more types of these vitamins. The contained amount of β-carotene and vitamins can be arbitrarily determined depending on the amount of each vitamin that is appropriate for administration by subjects including human. According to the recommendation by the American Heart Association, “healthy people should consume vitamins/minerals, anti-oxidative supplements from food not as supplement intake”. Therefore, preferably the vitamin preparation is not contained in the compositions for oral use, but can be contained when needed, regarding vitamin preparations contained in a composition for oral use.

The composition for oral use in the present invention is particularly preferably a tablet in light of the fact that the composition homogeneously contains eggshell membranes at high concentration, does not undergo changing in shape and/or disintegrating during storing, distributing, administering, has an excellent handling property, and is easily administered orally. Hereinafter, as the example of pharmaceutical composition using the activator of gene expression of molecular chaperone gene in the present invention, tablets are explained.

The amount of fine powder-like eggshell membrane component contained in the tablet in the present embodiment is not particularly limited. However, the amount of eggshell membrane component is preferably contained in a range from 5 to 40% by mass to the total mass of the tablet, more preferably in a range from 10 to 25% by mass in light of the perspective that granulation to particles and tablet production can be done smoothly, and gene activation effect of molecular chaperone is more superb and the reduction or scavenging activity of reactive oxygen species generated in the body increase when the tablet is orally administered (dosed).

When the amount of the eggshell membrane component is 5% by mass or more, the need for the administration of a large amount of tablets can be eliminated. When the contained amount of eggshell membranes in the tablets is adjusted 40% by mass or less, granulation to the particle and tablet production becomes easy and the tablets are easily produced.

In order to produce the tablets in the present embodiment, various additives, for example, bonding agents, disintegrating agents, lubricants, other nutritive components, and the like can be arbitrarily added in addition to diluting agents.

The diluting agent for producing tablets, at least one type of diluting agent such as modified starch and lactose is preferably used. The contained amount of the diluting agent is preferably in a range from 0.5 to 3 mass times with respect to the mass of the eggshell membrane component, more preferably in a range from 1 to 2.5 mass times in light of diluting property. Examples of modified starch include dextrin such as roasted dextrin (white dextrin, yellow dextrin, and the like), and the like; oxidized starch (hypochlorous acid oxidized starch and the like); and low-viscosity denatured starch (acid-immersed starch, enzyme-treated starch, and the like), and one or two or more of these substances can be used. In case modified starch (in particular, “Waxy a” and “pine fiber”) and lactose are concomitantly used as the diluting agent, the ratio for use of modified starch:lactose (mass ratio) is preferably in a range of 1:5 to 5:1, more preferably 1:3 to 3:1.

Publicly known bonding agents can be arbitrarily used as the bonding agent, for example, starch glue, gum arabic glue, hydroxypropyl cellulose, and the like can be recited.

Publicly known disintegrating agents can be arbitrarily used as the disintegrating agent, for example, celluloses and the like can be used. Moreover, starch has the function as a disintegrating agent.

Publicly known lubricants can be arbitrarily used as the lubricant, and for example, waxes such as magnesium stearate, sucrose fatty acid ester, and the like; and talc, vitamin C and the like can be recited.

Furthermore, the tablets in the present embodiment preferably contain eggshell calcium as an agent for improving hardness in order to increase the hardness of the tablet, to prevent deformation or brittleness of the tablets, and thereby to improve handling properties during packing, storing, delivering and the like, and to get excellent ingestion. Eggshell calcium is a fine powder prepared from pulverizing and drying the shell of eggs of avian species such as chicken and the like. Any eggshell calcium can be used for the tablet used in the present embodiment, as long as human can take the eggshell calcium. As eggshell calcium, for example, commercially available trade name “Calliope” from Kewpie Corporation that has been conventionally used, eggshell calcium produced by Taiyo Kagaku Co., Ltd., and the like can be used as they are. The contained amount of eggshell calcium in the tablet is preferably in a range from 5 to 20% by mass with respect to the total mass of the tablet, more preferably in a range from 8 to 15% by mass.

The tablets in the present embodiment are preferably coated with a coating film, for the purpose of preventing changes in properties or degradation of components containing in the tablets, or improving scratch resistance of the tablet surface. The coating film can be formed from the similar film-forming material that has been conventionally used as the coating film for tablets. As the film-forming material, for example, trade name of “Shellac” (track 30) by Gifu Shellac Manufacturing Co., Ltd. can be employed, but is not particularly limited to this.

Moreover, the tablets in the present embodiment are preferably coated with sugarcoat to be easily oral administered. Moreover, they may be colored when needed, and be subjected to polishing after coloring.

The size of the tablets in the present embodiment can be arbitrarily determined, but not particularly limited to this, preferably circular or oblong shape with about 7 to 10 mm in diameter in general from the points of view of handling properties and easiness of administration.

Furthermore, as the tablets in the present embodiment, for example, the weight of one tablet is preferably about 350 to 600 mg, and the eggshell membrane component is preferably contained about 18 to 240 mg per tablet, more preferably 35 to 150 mg. For example, it is presumed that the tablet of the present embodiment contains about 18 to 240 mg of the eggshell membrane component. In this instance, for adult, 1 to 200 tablets daily can be taken or administered (total amount of the eggshell membrane component of 18 to 48,000 mg daily).

The tablets in the present embodiment can be produced from a raw material containing at least fine powders containing eggshell membranes in the present embodiment by utilizing publicly known methods of producing tablets arbitrarily. Specifically, the tablet of the present embodiment can be produced at least conducting an uncoated tablet-producing process (tablet production process), which includes the process of using raw materials for tablet production and producing uncoated tablets. In addition to the uncoated tablet-producing process, processes such as a granulation process, a protective coating process, a sugarcoat coating process, and the like may be conducted. And further coloring, polishing, and the like may be conducted. The obtained tablets from these processes in the present embodiment go on the market after being subjected to sorting, measure, packaging, and the like.

### (Food Additives)

The activator of gene expression of molecular chaperone gene in the present invention can be used alone or by combining with various physiologically acceptable components such as other food additives as a food additive, in order to add to food products such as confectioneries, health foods, preserved foods, processed foods, and the like. The food additives of the present invention can be added to various food products by publicly known methods in the technical field of the art aiming at gene activation of molecular chaperone genes. For example, as the example of applicability of eggshell membranes to food products, a tablet or confectionery containing eggshell membranes that is ground in powder-like form is proposed (Japanese Patent No. 3862600, Japanese Patent Application Laid-Open No. 2009-165421). As the eggshell membrane powders used for tablets or confectioneries described in these patents, the food additive containing the activator of gene expression of molecular chaperone gene in the present invention can be used.

Herein, a “food product” is not limited to one for human, and includes feed for mammals such as dogs and cats that are reared as pets and domestic animals. Moreover, the concept of the “food product” includes beverages, so-called supplements, health foods, enteral nutrition foods, special-use foods, foods with nutrient function claims, foods for specified health use as well as general food products.

### EXAMPLES

Hereinafter, the present invention is explained by Examples, however, the present invention is not limited to the following Examples.

1. Production of an External Preparation containing an Eggshell Membrane Hydrolysate

As an alkali-hydrolyzed form of eggshell membranes (hereinafter referred to as “ASESM”), “EMPROTEIN-P”, the trade name of eggshell membrane, obtained from Kewpie (Kewpie Corporation, Tokyo, Japan) was used. The relative molecular weight of the main part of this ASESM was in the range from 12 to 14 kDa by measurement using size exclusion chromatography (gel filtration) (NPL 1: Ohto-Fujita et al, Cell Tissue Res. 2011 July; 345(1): 177-190).

A solution (lotion) containing 10% (W/V) of ASESM was produced by using bases containing 7% (V/V) of butylene glycol, 1% (V/V) of pentylene glycol, 4% (V/V) of glycerin, and a 0.2% (V/V) of a phenoxyethanol aqueous solution.

### 2. Effect of ASESM External Preparation on Gene Expression of Molecular Chaperone Gene in the Dorsal Region of a Hairless Mouse

Hairless mice (Hos: HR-1, 6 weeks old, male) were used as experimental animals (control group: n=2, a group treated with ASESM: n=2). For the group of treating with ASESM, 10% (W/V) of said ASESM solution prepared above was applied externally (topically) to the dorsal region of the skin for 10 days (40 μl/time×2). As the control group, the above-mentioned base solution that did not contain ASESM was applied in the similar manner.

Analysis of quantitative real-time PCR (quantitative real-time polymerase chain reaction) data was carried out as follows. A skin sample was taken from each mouse and ground it in liquid nitrogen. After homogenizing the whole skin tissue, the total RNA was isolated by using “TRIzol (registered trademark) Reagent”, the trade name of solution. The total RNA (200 ng) was applied to cDNA synthesis using “Takara PrimeScript RTR reagent kit”, the trade name of a kit. The real-time PCR method was carried out by using “SYBRR Premix Ex Taq™ II (Takara) on Thermal Cycler Dice Real Time System” (Takara), the trade name of reagent. Primers that were designed to amplify genes encoding Hspb2, Hspb5 (Cryab), Hspa1b (Hsp70-1), Hspd1 (Hsp60), Hsp90aa1, Hsp90ab1, Hsp90b1, and Trap1 were used as the primer. As the internal standard, glyceraldehyde-3-phosphate dehydrogenase (GAPDH) mRNA, a housekeeping gene, was amplified in the same manner. PCR cycle was used in the following regimen of thermal cycling: the first denaturing was carried out at a temperature of 95° C. for 30 seconds, and then 40 cycles of the amplification was carried out (denaturing at a temperature of 95° C. for 5 seconds, and annealing and elongation reaction at a temperature of 60° C. for 1 minute). The primer used is summarized in the following Table 1.

TABLE 1

| Se- | | Gene Bank | | | | Ampli- |
|---|---|---|---|---|---|---|
| quence | Subject for | registry | | | | con |
| Number | amplification | number | Primer | Sequence (5′→3′) | Length | size |
| 1 | Hsp beta-2 isoform | NM_024441 | Mm_Hspb2-F | TCGAGCTGCTCTATCCCATGAT | 22 | 171 |
| 2 | 1 {Mus musculus} | NM_001164708 | Mm_Hspb2-R | GGGTCTATCGCTGGCAGTCA | 20 | |
| 3 | Mus musculus | NM_009964 | Mm_Cryab-F | GACCTCTTCTCAACAGCCACTTC | 23 | 113 |
| 4 | crystalin, alpha B | | Mm_Cryab-R | TCCTTCTCCAAACGCATCTCTGA | 23 | |
| | (Cryab) | | | | | |
| 5 | Mus musculus Hsp 1B | NM_010478 | Mm_Hspa1b-F | TCTTGGCACTGTGTACTACATAACT | 25 | 139 |
| 6 | (Hspa1b) | | Mm_Hspa1b-R | GCAGGAAGGATACACCATCTTTAAC | 25 | |
| 7 | Mus musculus Hsp 1 | NM_010477 | Mm_Hspd1-F | GGTTGGAAGAAAGGGTGTCATCA | 23 | 200 |
| 8 | (chaperonin) | | Mm_Hspd1-R | GGACAATGGACTGAACACTGGAA | 23 | |
| | (Hspd1) | | | | | |
| 9 | Mus musculus Hsp 90, | NM_010480 | Mm_Hsp90aa1-F | GGACTGACACAGGTGAACCAATG | 23 | 169 |
| 10 | alpha (cytosolic), | | Mm_Hsp90aa1-R | TCCTTATCTCGTTCCTTCTCCACA | 24 | |
| | class A member 1 | | | | | |
| | (Hsp90aa1) | | | | | |
| 11 | Mus musculus Hsp 90, | NM_008302 | Mm_Hsp90ab1-F | ATTGACCAGGAGGAGCTGAACA | 22 | 147 |
| 12 | alpha (cytosolic), | | Mm_Hsp90ab1-R | ACCTTCTACAGAGAAGTGCTTGAC | 24 | |
| | class B member 1 | | | | | |
| | (Hsp90ab1) | | | | | |
| 13 | Mus musuculus Hsp 90 | NM_011631 | Mm_Hsp90b1-F | GTGGAGTAGCAAGACAGAGACTG | 23 | 168 |
| 14 | beta (Grp94), member | | Mm_Hsp90b1-R | TTCATAAGTTCCCAATCCCACACA | 24 | |
| | 1 (Hsp90b1), | | | | | |
| 15 | Mus musculus TNF | NM_026508 | Mm_Trap1-F | GGAGATCAACCCCAGGCACA | 20 | 114 |
| 16 | receptor-associated | | Mm_Trap1-R | GCTGCTATCATGGCATTCTCATAG | 24 | |
| | protein 1 (Trap 1) | | | | | |

The level of each gene expression was calculated as the ratio with respect to the GAPDH expression level. The results are shown in Table 2.

TABLE 2

| | Promoting effect of gene |
|---|---|
| | expression compared to |
| Gene | control |
| Hspb5 (Cryab) | P = 0.077 ↑ 4.6 times |
| Hspa1b | P = 0.257 ↑ 9.2 times |
| (Hsp70-1) | |
| Hspd1 (Hsp60) | P = 0.437 ↑ 1.4 times |
| Hsp90aa1 | P = 0.033 ↑ 5.0 times |
| Hsp90ab1 | P = 0.185 ↑ 6.6 times |
| Hsp90b1 | P = 0.120 ↑ 26.5 times |
| Trap1 | P = 0.155 ↑ 9.7 times |

From these results, in the dorsal region of the skin in the ASESM treatment group, the gene expression of various molecular chaperone genes was controlled and the expression levels of a plurality of molecular chaperone genes were remarkably promoted at the same time as the result of the action of ASESM to the cells.

3. Production of Fine Powders containing Eggshell Membranes

As a sample of powders containing eggshell membranes, a product under the trade name of “EM powder 300” by Kewpie Corporation was ground with a jet mill and the ground powders were used. As the jet mill, using a single track jet mill (Seishin Enterprise Co., Ltd., FS-4), grinding was carried out under the condition of 1.2 m³/min of air volume, 11 kw of dynamic power, until the maximum volume particle diameter reached approximately 800 mesh (opening was about 20 μm). The maximum volume particle diameter of the fine powders containing eggshell membranes was 19.6 μm and the mean volume particle diameter of them was 5.8 μm after grinding by measurement using a laser diffraction particle size distribution analyzer (Seishin Enterprise Co., Ltd., LMS-30).

4. Effect of the Administration of the Powders containing Eggshell Membranes on the Gene Expression Level of Molecular Chaperone Gene in Mice

Male Hos/HR-1 mice at 8 weeks old had been fasted one day before the administration. 0.5 mg of a supplement for experiment containing only eggshell membrane fine powders and eggshell calcium as active ingredients (“8φCR 200 mg”, fine powders containing eggshell membranes produced in the above-mentioned section (800 mesh) 37.50% (75.0 mg); eggshell calcium (Kewpie Corporation) 11.75% (23.5 mg); lactose (Granbia plc) 43.75% (87.5 mg); corn protein (Kobayashi Perfumery Co., Ltd.) 5.00% (10.0 mg); hydrogenated rapeseed oil (Kawaken Fine Chemicals Co., Ltd) 2.00% (4.0 mg)), or 0.5 mg of a control tablet containing only a diluent agent as a control (“9φCR 250 mg”, lactose 93.00% (232.5 mg); corn protein 5.00% (12.5 mg); hydrogenated rapeseed oil 2.00% (5.0 mg)) (the tablet ground to powders with a mortar) was suspended in a jelly for animal drug administration (a trade name, MediGel Sucralose, Japan SLC, Inc.) at a volume of 100 μL. The full dosage of these suspensions was directly administered to the stomachs of the mice by using a sonde under light ether anesthesia (n=3 in each group). Mice were anatomized 8 hours after administration, the gene expression levels of Hspb5 and Hspb2 genes in cells of the skin tissues were evaluated by quantitative real-time PCR in the same manner as the above description.

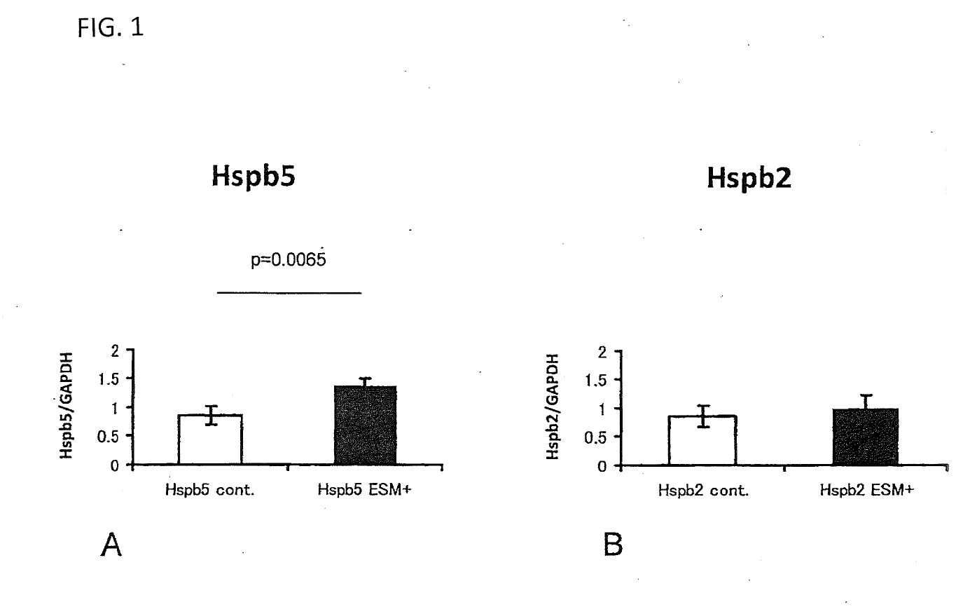

The result shows in FIG. 1. In the group that the powders containing eggshell membranes were administered, the expression levels of both Hspb5 (Cyrab) and Hspb2 inclined to increase. However, since the data obtained from real-time PCR is a comparative assessment, the values on the vertical axis vary depending on which expression level is determined as 1. The point to note is the difference in the data between experiment and control experiment.

### 5. Disposition of an Eggshell Membrane Component

When nitrogen-containing compounds such as proteins are mixed with lithium carbonate and are radiated by neutron radiation, they are labeled by tritium generated through the reaction of Li⁶(n,α)³H. By utilizing this phenomenon, the disposition of the tritium-labeled powders containing eggshell membranes was studied as follows when they were orally administered to the mice.

### <Labeling of Eggshell Membranes>

The powders containing eggshell membranes at the amount of 0.32 g (“EM powder”, Kewpie) and lithium carbonate at the amount of 0.65 g were well mixed and the mixture was encapsulated into a silica tube under reduced pressure, and then was irradiated by neutrons for 20 minutes at the Japan Atomic Energy Agency, Nuclear Research Institute (JRR4 nuclear power reactor). The irradiated sample was taken out of the silica tube and mixed with water to dissolve unreacted lithium carbonate. Eggshell membrane powders are water-insoluble, so it was recovered by filtration. And the filtrate was washed with water until the radioactivity of the filtrate was sufficiently reduced to remove tritium unbound to the eggshell membranes.

### <Experimental Animals>

C57BL/6J mice at 6 weeks old purchased from Oriental Yeast Co., Ltd. were underwent about one-week of preliminary rearing (at a temperature of 23±2° C., relative humidity 55±10%, under the environment of 12-hour light-dark cycle), and then experiments were carried out when the mice reached to 7 weeks old. Mice were reared in a metabolism cage (Metabolica MM, SUGIYAMA-GEN CO., LTD.,) (86.5 cm²×14.5 cm, space approximately 2000 cm³) individually, and were taken solid feed (MF, Oriental Yeast Co., Ltd.) and tap water ad libitum.

### <Modes of Administration>

The labeled powders containing eggshell membranes suspended in water were forcedly administered once in the stomach orally by using a plastic disposal sonde to mice that were on fasting for 16 hours before the administration of the above the labeled powders. The radioactivity of the applied dose was about 4.5 MBq/kg (122 mCi/kg) body weight, the treatment dosage was 250 mg/kg body weight.

### <Measurement of Radioactivities>

A scintillator solution was added to the prepared samples for measurement of radioactivities, and the radioactivities were measured with a liquid scintillation counter (Packard, 2200CA). The correction of quenching was carried out according to an external standard channel ratio method.

### <Measurement of Radioactivity Concentrations in Blood>

Blood samples at the volume of 5 ml were collected from the tail veins of the mice 0.25, 0.5, 1, 2, 4, 6, 9, 12, 24 hours and 2, 3, 4, 5, 6 days after the administration of the labeled powders containing eggshell membranes. To these samples were added 1 ml of a tissue solubilizer (Soluene-350 (Perkin Elmer)/isopropyl alcohol (1 1)), and the mixture was warmed and shaken at a temperature of 50° C. for 3 hours. After the warming and shaking, 500 ml of a 30% hydrogen peroxide solution was added. To this sample was added 10 ml of a scintillator (Hionic fluor, Perkin Elmer), and the radioactivities were measured.

<Elimination of Radioactivities into Urine/Feces>

After administrating the labeled compound, mice were placed in the metabolism cage(s) (Metabolica MM, SUGIYAMA-GEN CO., LTD.,), and then urine and feces were collected every day for 6 days after each administration. Some of the collected feces samples were accurately measured and 2 ml of the tissue solubilizer was added to the samples, and the mixture was warmed at a temperature of 50° C. for 3 to 4 hours. Thereafter, 1 ml of isopropanol was added to the mixture, and the mixture was further warmed at a temperature of 50° C. for 2 hours. To this sample were added 0.5 ml of a 30% hydrogen peroxide solution and 10 ml of scintillator (Hionic Fluor, Perkin Elmer), and then the radioactivities were measured. For urine samples, 5 ml of a scintillator (Ultima Gold LLT) was added to 1 ml of each fraction, and then its radioactivity was measured.

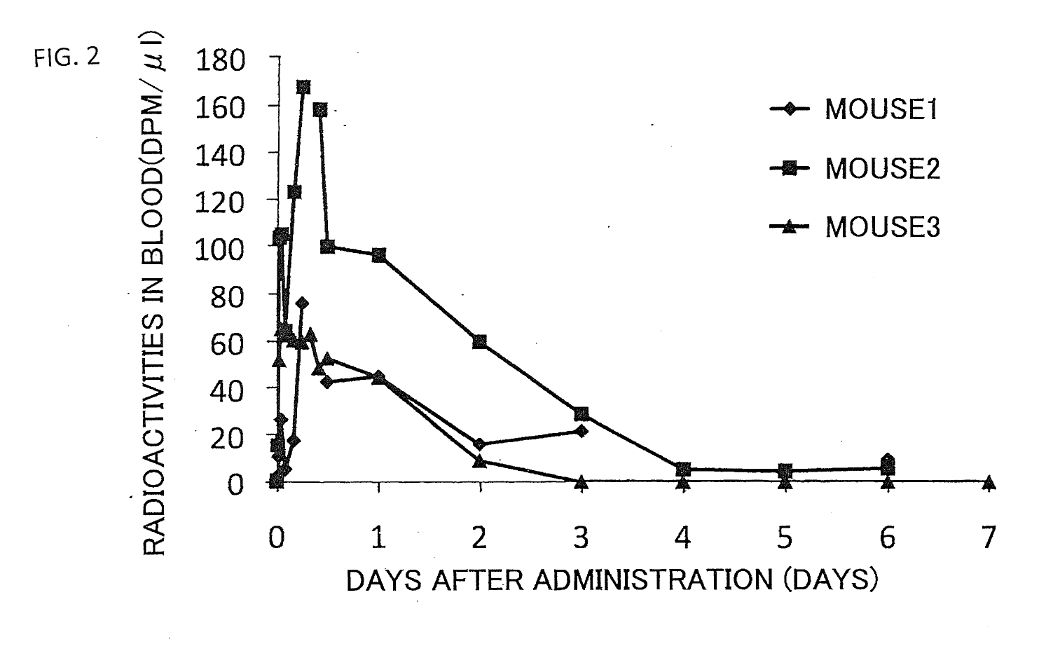

The result of radioactivity concentrations in the blood after the oral administration of the eggshell membranes to the mice shows in FIG. 2. The radioactivity concentrations in the blood over the time after the administration of the tritium-labeled eggshell membranes were shown. The radioactivity concentrations in the blood reached to the maximum within 24 hours after administration, and decreased to the original levels of radioactivity in 3 to 4 days.

The result of radioactivity concentrations in feces/urine shows in Table 3. The radioactivity in feces/urine was 61.05% of the total amount of administration by 3 days after administration.

TABLE 3
³H radioactivity (ratio with respect to the dosage of
administration (%))

| | Day 1 | Day 2 | Day 3 |
|---|---|---|---|
| In feces | 14.78 | 33.02 | 11.93 |
| In urine | 0.47 | 0.50 | 0.35 |

### <Distribution of the Radioactivities to Tissues of the Body>

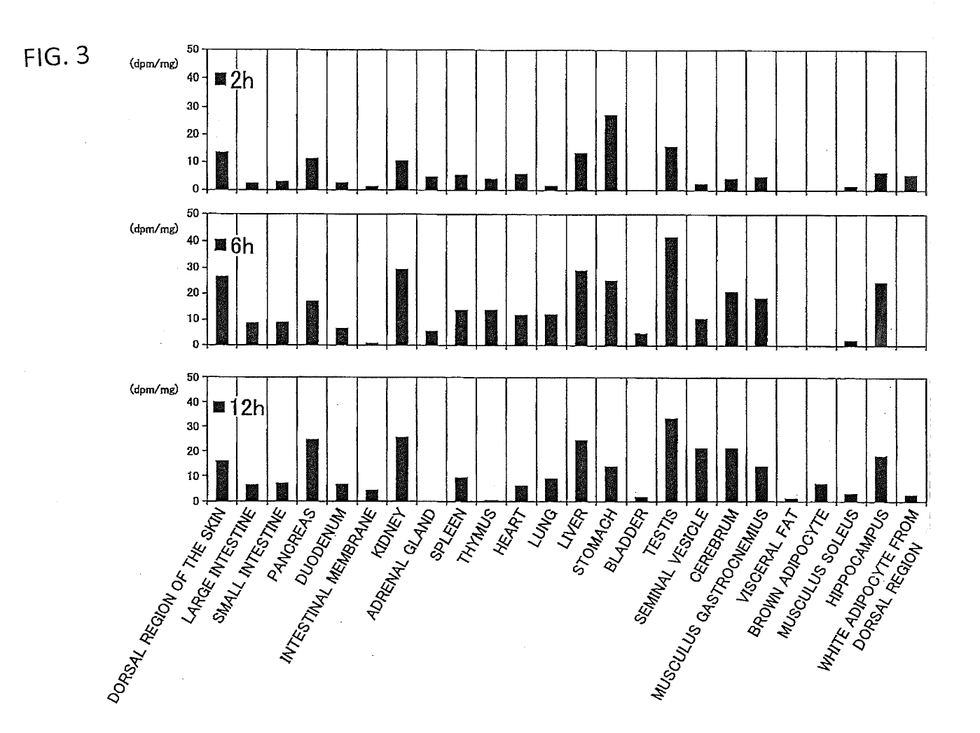

The tritium-labeled eggshell membranes were administered at the radioactivity of 5,568,000 dpm to the inside of the stomach of three mice by using the sonde in the similar manner as previously described. Part of or the whole tissues were extracted from each individual 2 hours, 6 hours, and 12 hours after administration and the weight thereof was measured. To each tissue was added 2 ml of a solubilizer (Soluene-350) and the mixture was incubated at a temperature of 60° C. for 3 hours. To this sample was added 0.5 ml of a 30% hydrogen peroxide solution and 10 ml of scintillator (Hionic Fluor), and the mixture was incubated at room temperature for 1 hour, and then the radioactivities were measured with a liquid scintillation counter. The results show in Table 4 and FIG. 3.

TABLE 4

| | 2 h | 6 h | 12 h |
|---|---|---|---|
| Dorsal region of the skin | 14.39827957 | 26.5335 | 16.06 |
| Large intestine | 3.423908524 | 8.58844221 | 6.5 |
| Small intestine | 4.218064516 | 8.83794393 | 7.12 |
| Pancreas | 12.13833922 | 16.8112288 | 24.69 |
| Duodenum | 3.623255814 | 6.47457627 | 6.57 |
| Intestinal membrane | 2.345011601 | 0.75178571 | 4.33 |
| Kidney | 11.43458498 | 29.500316 | 25.59 |
| Adrenal gland | 5.806666667 | 5.34285714 | 0 |
| Spleen | 6.379558011 | 13.558216 | 9.46 |
| Thymus | 5.085465116 | 13.5094737 | 0.18 |
| Heart | 6.943851508 | 11.6870663 | 5.93 |
| Lung | 2.68342246 | 11.8900826 | 8.89 |
| Liver | 14.24795918 | 28.8658174 | 24.23 |
| Stomach | 27.68076923 | 24.9255708 | 13.85 |
| Bladder | 0 | 4.62395833 | 1.5 |
| Testis | 16.60234375 | 41.5360656 | 33.31 |
| Seminal vesicle | 3.254893617 | 10.1778351 | 21.19 |
| Cerebrum | 5.092879257 | 20.5594406 | 21.05 |
| Musculus gastrocnemius | 5.785743381 | 18.1498305 | 13.87 |
| Visceral fat | 0 | 0 | 0.8 |
| Brown adipocyte | 0 | 0 | 6.79 |
| Musculus soleus | 2.421428571 | 1.84 | 2.73 |
| Hippocampus | 7.248201439 | 24.0738462 | 17.88 |
| White adipocyte from | 6.22173913 | 0.03995215 | 2.21 |
| dorsal region | | | |

It was found that the large quantity of the eggshell membrane component was distributed in various tissues throughout the body, in particular the skin, kidney, liver, testis (ovary in female), and brain (for example, hippocampus). Accordingly, the eggshell membrane component is expected to show the stimulatory effect on the expression levels of molecular chaperone genes in these tissues.

### 6. Production of a Pharmaceutical Composition (Tablets)

### (1) Production of Granules for Tableting

A raw material was prepared by mixing fine powders containing 20.0 pts. mass of eggshell membranes produced in the process mentioned above (800 mesh); 10.0 pts. mass of “Waxy a” by produced Nisshoku Co., Ltd.; 20.0 pts. mass of “pine fiber” produced by Matsutani Chemical Industry Co., Ltd.; 25.9 pts. mass of lactose (Meggle Japan Co., Ltd.); 10 pts. mass of eggshell calcium (“Calhope” produced by Kewpie Corporation); 5.0 pts. mass of β-carotene; 20.05 pts. mass of vitamin B; 0.05 pts. mass of vitamin E; and 2.0 parts niacin with a V-type mixing machine. Subsequently, 15 pts. mass of ethyl alcohol was mixed with 93.0 pts. mass of this raw material mixture, and the mixture obtained from this procedure was ground with a wet granulation equipment. And then, the mixture was dried at a temperature of 50° C. for approximately 16 hours to produce granules for tableting.

### (2) Tableting

Next, 9 pts. mass of vitamin C and 1 pt. mass of sucrose fatty acid ester were mixed with respect to 100 pts. mass of granules for tableting, and uncoated tablets weighing 200 mg/tablet were produced from the resultant mixture with an equipment for tablet production.

### (3) Protective Coating

Subsequently, an aqueous solution of “Shellac” by Gifu Shellac Manufacturing Co., Ltd. was applied to the surface of the uncoated tablets with a coating equipment. And then the uncoated tablets were dried at a temperature of 40° C. for 2 hours to obtain tablets with protective coating (protective-coated tablets).

### (4) Sugarcoat Coating

The protective-coated tablets were completely dried and the surfaces of the protective-coated tablets were coated with a paste A for sugarcoat (a paste obtained from mixing 70 pts. mass of granulated sugar, 3 pts. mass of gum arabic, 4 pts. mass of gelatin, 3 pts. mass of eggshell calcium and 65 pts. mass of water) with a sugarcoat coating equipment, and then the tablets were dried at a temperature of about 40° C. for 4 hours. Thereafter, water was added to the paste A for sugarcoat to obtain a diluted paste B for sugarcoat. Furthermore, the paste B for sugarcoat was coated onto the surface of the tablets, which were treated with the paste A for sugarcoat and dried, with a sugarcoat coating equipment, and then the tablets were dried at a temperature of about 40° C. for 4 hours. From this process, tablets coated with the paste for sugarcoat (sugarcoated coating tablets) were obtained.

### (5) Coloring

A coloring solution containing “SR Red K3” by San-Ei Gen F.F.I., Inc. was applied to the surfaces of the sugarcoated coating tablets, and then the tablets were dried at a temperature ranging from 40 to 50° C. for 4 hours to produce red-colored tablets (colored tablets).

### (6) Polishing

Polishing was undertaken on the surfaces of the colored tablets using Carnauba wax. The mass of the obtained tablet in this process was 400 mg per tablet, namely approximately 40 mg of the eggshell membrane component was contained per tablet.

### (7) Sorting-Measure-Packaging

After polishing the tablets, defective products were sorted out, and the tablets were measured their weights after the inspection of products, and packaged with double-layered pouch(es) enclosing desiccants. The tablets had sufficient hardness, shape retention properties, and excellent handling properties without changing in shapes or deteriorating during sorting, inspection, and packing.

The embodiments and Examples of the present invention were explained above, however, the present invention can be modified in various ways within the spirit and scope of the present invention. For example, one embodiment of the activator of gene expression of molecular chaperone gene in the present invention can be utilized in other intended purposes such as regenerative medicine (stem cell, iPS cells, and the like), materials for base, and the like other than compositions for oral use, compositions for external use, food products (supplements), food additives, and the like. Moreover, the powder containing eggshell membranes used for this purpose is preferably a fine powder having 6 μm or less in the mean volume particle diameter, and/or 20 μm or less in the maximum volume particle diameter. However, the mean volume particle diameter may be made to exceed 6 μm or the maximum volume particle diameter may be made to exceed 20 μm.

Moreover, Hspb5 (Cryab), Hspa1b (Hsp70-1), Hspd1 (Hsp60), Hsp90aa1, Hsp90ab1, Hsp90b1, Trap1 and Hspb2 were selected as the genes encoding molecular chaperones, however, the other genes encoding molecular chaperones may be added. Also, other genes encoding molecular chaperone may be added instead of one or more of the genes mentioned above, one or a plurality of genes may be selected from the new combination thereof as the genes encoding molecular chaperones.

For examples, It is good also as any one or the plurality of the other type of HSP (heat shock protein/stress protein/molecule), endoplasmic reticulum stress response related gene (UPR related gene), and Protein degradation related gene to add to Hspb5 (Cryab), Hspa1b (Hsp70-1), Hspd1 (Hsp60), Hsp90aa1, Hsp90ab1, Hsp90b1, Trap1 and Hspb2 or replace with them.

The other type of HSP are as follows; Hsp104, Hsp110, Hsp90, Grp94, Hsp70, Hsp71, Hsp72, Grp78, Hsx70, Hsp40, Hsp60, Small HSPS: Hsp27(HspB1, Hsp25), myotonic dystrophy protein kinase-binding protein (MKBP) (HspB2), HspB3, αA-crystallin (HspB4), αB-crystallin (HspB5), Hsp20 (p20, HspB6), cardiovascular heat shock protein (cvHsp [HspB7]), Hsp22 (HspB8), HspB9, sperm outer dense fiber protein (ODFP, HspB10).

The other type of HSP areas follows; HSPA6Hsp70, X51757 (hypothetical protein), SERPINH1 Hsp47 (serpin peptidase inhibitor), HSPA1A (Hsp70), SERPINE1 (serpin peptidase inhibitor), BAG3 (BCL2-associated protein), HSPA1B (Hsp70), BG537255 (hypothetical protein), DNAJB1 (DnaJ, Hsp40), FLJ12587 (BTB/POZ KELCH domain protein), BE328496, RFPL2 (putative Ret finger protein), IER5 (regulator of immediate early response), APBA3 (amyloid beta precursor-binding protein), HSPA1L (Hsp70), HSPH1 (Hsp105/110), MBNL2 (muscleblind-like protein), HMOX1 (Heme oxygenase), DOK2 (docking protein), HSPB1 (Hsp27), DNAJB6 (DnaJ, Hsp40), FBXL14 (F-box and leucine-rich repeat protein), HSPA4L (Hsp70), DNAJA1 (DnaJ, Hsp40), MS4A6A (membrane-spanning), CLDN15 (claudin), PRKACB (protein kinase), APG5L (involved in autophagy), MAST3 (serine/threonine kinase), BF982927 (hypothetical protein), SNAP23 (synapto-somal-associated protein), TNFSF14 (ligand of tumor necrosis factor), DBP (albumin promoter binding protein), TX35NIP (thioredoxin interacting protein), RGS2 (regulator of G-protein signalling), TJP4 (tight junction associated protein), SCML1 (sex comb on midleg-like), MINPP1 (multiple inositol poly-phosphate histidine phosphatase), NXT2 (nuclear transport factor), MRPL18 (mitochondria 1 ribosomal protein), MRPS6 (mitochondria 1 ribosomal protein), MTRF1 (mitochondria 1 translational release factor), GNA15 (guanine nucleotide binding protein), PTDSR (phosphatidyl serine receptor), ULBP2 (UL16 binding protein), DUSP1 (dual specificity phosphatase), NUCB1 (nucleo-binding protein), RFPL3 (Ret finger protein-like), ZC3HA V1 (zinc finger protein CCCH-type, antiviral), PRNP (prion protein, p27-30), IL16 (interleukin), TRIM26 (tripartite motif-containing protein), TGFA (transforming growth factor), CHN2 (chimerin), SGK (serum/glucoc orticoid regulated kinase), FKBP4 (FK506 binding protein), FXR1 (putative fragile X mental retardation protein), SCARB2 (scavenger receptor), ZFX (zinc fingerprotein), RPS6KA4 (ribosomal protein kinase), CDC6 (cell division cycle 6 homolog), GPR89 (G protein-coupled receptor), IL2RB (interleukin receptor), GPR18 (G protein-coupled receptor), STCH (Hsp70), ZNF473 (zinc finger protein), PELI2 (pellino homolog), DRE1 (Dre1), LENG4 (leukocyte receptor cluster protein), CASP1 (caspase, cysteine protease), AC004522 (hypothetical protein), CHIC2 (cysteine-rich hydrophobic domain), FAS (TNF receptor), STIP1 Sti1 (Stress-induced-phospho-protein, Hsp70/Hsp90-organizing protein), PHACTR1 (phosphatase and actin regulator), LTBP1 (latent transforming growth factor beta binding protein), CACYBP (calcyclin binding protein), SLC38A2 (amino acid transporter), ARID4A (AT rich interactive domain containing protein), AL023584 (hypothetical protein), ACTN1 (actinin), ENC1 (ectodermal-neural cortex), SEPW1 (seleno-protein), SIPA1L3 (signal-induced proliferation-associated), SPAG5 (sperm associated antigen), CHORDC1 (cysteine and histidine-rich domain), GOLPH3L (golgi phosphor-protein like), HSPA4 (Hsp70), RHOH (Ras homolog), ACA T2 (acetyl-CoA acetyltransferase), ADFP (adipose differentiation-related protein), FLJ21865 (endo-beta-N-acetylglucosa minidase), ChGn (chondroitin beta1,4 N-acetyl-galactos-aminyl-transferase), PREI3 (preimplanta-tion protein, kinase activator), PTPN12; PTPG1 (tyrosine phosphatase), TES (testis derived transcript, hypothetical protein), CSIG (ribosomal L1 domain), AHSA1 (activator of Hsp90), BANP (nuclear protein), AFFX-M27830_M, CDKN2C (cyclin-dependent kinase inhibitor), IER3 (involved in immediate early response regulation), NEDD4L (ubiquitin-protein ligase).

Endoplasmic reticulum stress response related gene (UPR related gene) are as follows; Sec61α,β,γ and TRAM, calnexin/calreticulin, ERp57, 1,2-mannosidase-like protein (EDEM), ER-associated degradation (ERAD), and Alzheimer's disease: Presenilin 1, PERK, eIF2α, BiP, Parkinson's disease: Parkin, α-synuclein, Amyotrophic lateral sclerosis (ALS): Cu/Zn-superoxide dismutase (SOD), ASK1 and Derlin-1, Schizophrenia: XBP1, Stroke: PERK-eIF2α, ASK1, CHOP, Heart disease: IRE1, PERK-eIF2α, ASK1, Atherosclerosis: IRE1, JNK, TRAF2, XBP1, Type 1 diabetes mellitus: IRE1, JNK, TRAF2, XBP1, Type 2 diabetes mellitus: XBP1, JNK Cancer: BiP, XBP1, ATF6, PERK, Autoimmune disease: XBP1, HLA-B27, and Glomerulonephritis, acute kidney injury: BiP, CHOP, Bc12

Protein degradation related gene are as follows; Proteasomes system: 26S proteasome, immunoproteasome, ubiquitin-proteasome system, ubiquitin ligase (E3), and LC3 (light chain 3)/GABARAP (gamma-aminobutyric acid receptor-associated protein), and Autophagy system: mTOR, AMPK, p53.

1. A method of activating gene expression of a molecular chaperone gene in a subject in need thereof comprising the step of administering to the subject an effective amount of a composition comprising a powder containing eggshell membrane or a soluble component of eggshell membrane.

2. The method according to claim 1, wherein the method comprises administering the composition topically, and wherein the composition further comprises a diluting agent.

3. The method according to claim 2, wherein the composition comprises a soluble component of eggshell membrane.

4. The method according to claim 3, wherein the soluble component of eggshell membrane is a hydrolysate of the eggshell membrane.

5. The method according to claim 4, wherein the composition further comprises one or more active ingredients selected from the group consisting of antiphlogistic agents, anti inflammatory drugs, melanin production suppressants, melanin reducing agents, hair bleaching agents, melanin excretion promoters, cell activators, antioxidants, anti-oxidizing agents, keratolytic-release agents, sebum suppressors, moisturizing agents, emollient agents, sebum secretion suppressors/promoters, ultraviolet absorbing agents, antiperspirants, blood circulation improving agents, exfoliating cleansers/softening agents, skin whitening agents, anti allergic drugs, steroid hormones, immunosuppressant, and antibiotics.

6. The method according to claim 1, wherein the method comprises administering the composition orally, and wherein the composition further comprises a diluting agent.

7. The method according to claim 6, wherein the composition comprises a powder containing eggshell membrane.

8. The method according to claim 7, wherein the powder containing eggshell membrane has a mean volume particle diameter of about 5 μm to about 40 μm.

9. The method according to claim 6, wherein the composition is in the form of a tablet and comprises one or more of bonding agents, disintegrating agents, lubricants, and nutritive components.

10. The method according to claim 9, wherein the diluting agent is modified starch or lactose.

11. The method according to claim 7, wherein the diluting agent is present in an amount of 0.5 to 3.0 mass times the powder containing eggshell membrane.

12. A method of enhancing the expression of one or more genes selected from the group consisting of Hspb5 (Cryab), Hspa1b (Hsp70-1), Hspd1 (Hsp60), Hsp90aa1, Hsp90ab1, Hsp90b1, Trap1, and Hspb2 in a subject in need thereof, comprising the step of administering to the subject an effective amount of a composition comprising a powder containing eggshell membrane, or a soluble component of eggshell membrane.
